(19) United States
(12) Patent Application Publication
(10) Pub. No.: US 2018/0333109 A1
(43) Pub. Date: Nov. 22, 2018

(54) CONTRAST ENHANCED ENERGY SUBTRACTION MAMMOGRAPHY

(71) Applicant: Robert Zamenhof, Brookline, MA (US)

(72) Inventor: Robert Zamenhof, Brookline, MA (US)

(21) Appl. No.: 15/983,656

(22) Filed: May 18, 2018

Related U.S. Application Data
(60) Provisional application No. 62/508,159, filed on May 18, 2017.

(51) Int. Cl. A61B 6/02; A61B 6/00

(52) U.S. Cl. CPC A61B 6/025; A61B 6/0414; A61B 6/482; A61B 6/502

(57) ABSTRACT

Disclosed are systems and methods for X-ray imaging of a patient's breast using contrast-enhanced energy-subtraction mammography employing a molybdenum-based contrast agent.

### RELATED APPLICATIONS

This application claims the benefit of priority to U.S. Provisional Patent Application Ser. No. 62/508,159, filed May 18, 2017.

### BACKGROUND OF THE INVENTION

Early detection and diagnosis of breast cancer is essential for successful treatment. In the United States breast cancer accounts for 32% of cancer incidence in women and 18% of cancer deaths (1). This translates to 40,600 female breast cancer deaths per year. Therefore, any improvement in early breast cancer diagnosis would have a major impact on public health.

Currently, screening mammography (i.e., routine mammographic exams of the adult female population) is done using dedicated mammography X-ray equipment that provides options for X-ray exposure using molybdenum, rhodium, or tungsten X-ray spectra. Screening mammography is intended to identify subjects who do not have breast cancer, but if a diagnosis is either ambiguous or positive, ‘diagnostic’ mammography is done, which invokes additional tools for assessing the breast, such as spot-compression or magnification mammograms, breast MRI with gadolinium contrast (2), or ultrasound (3).

Another recent advance in X-ray screening mammography has been digital breast tomosynthesis (DBT) (4). In this procedure, X-ray images of the breast are obtained without contrast by acquiring 15-20 individual mammographic exposures of the breast over an angular sweep of 15-20 degrees, followed by reconstruction of the data into pseudo-3D image planes through the breast. Observing the X-ray image of the breast by ‘paging’ through the reconstructed parallel planes aids the mammographer in visualizing overlapping glandular tissue as separate layers such that there is less likelihood of lesions being obscured. This technique has been shown to improve screening accuracy over conventional mammography, particularly in patients with large and/or dense breasts.

In an effort to address the challenge in X-ray imaging of the breast in differentiating breast cancer from benign abnormalities, consideration has been given to contrast-enhanced dual-energy imaging. In contrast-enhanced imaging, a contrast agent that may be iodine-based is introduced into the breast, typically through an intravenous catheter placed in a brachial vein, after which X-ray images are taken, commencing with a pre-injection image, followed by a post-injection image when the contrast agent has distributed itself within the breast microvasculature. The contrast agent helps highlight the microvascular architecture in the breast, which, in turn, may highlight breast cancer. If X-ray images taken before and after the contrast agent has distributed itself among the microvasculature of normal breast tissues and tumor are subtracted from each other, the architecture of the breast microvasculature may be visualized more clearly because the subtraction process suppresses the confounding normal breast tissue architecture—or ‘clutter’ as it is often referred to. This, in turn, may assist in differentiating cancer from benign breast tissue, since there is credible evidence that angiogenesis factors modulate the formation of abnormal microvasculature around and within breast cancers. It is speculated that the growth of breast cancer is modulated by angiogenesis, and that the resulting neovasculature manifests increased permeability and a tortuous appearance. Thus, an X-ray technique that is able to image such neovasculature as well as the regional X-ray opacification due to its increased permeability, would have the potential of high sensitivity for the detection of breast cancer.

Contrast-enhanced MM (CMRI) may detect breast cancers by imaging the neovasculature and the consequences of microvascular permeability associated with breast cancers. Although CMRI has a high sensitivity for detecting breast cancer it nevertheless has disadvantages that include high cost, long procedure time, enhancement of benign abnormalities such as fibroadenomas, leading to poor specificity, and precludes the imaging of women with surgical metal clips or insurmountable claustrophobia. Typically, the contrast agents used in CEMRI are gadolinium-based and are different from the iodine-based contrast agents used in X-ray imaging.

X-ray mammography can also benefit from contrast enhancement in order to improve cancer detection. Currently, the only contrast agents used in breast imaging are iodine-based. X-ray methods used in imaging the breast for the detection of breast cancer include breast CT, breast, breast tomosynthesis, and digital mammography. Contrast-enhanced mammography (CEM) has been shown to improve the detection rate of breast cancers (6). It has also been suggested that CEM may provide improved specificity compared to CEMRI. At this time, however, such studies are sparse and may need to be validated with larger clinical trials.

Contrast-enhanced mammography has been evaluated using two distinct methods. The first involves subtraction of images obtained at single X-ray energies pre- and post-contrast administration. This method is referred to as “temporal subtraction imaging.” The second method involves subtraction of images taken in rapid succession at two different X-ray energies, and is referred to as “dual-energy subtraction imaging.” In the latter method, images are obtained at low and high X-ray energies following the administration of an iodine-based contrast agent. These images are acquired with X-ray spectra shaped to correspond to regions above and below the k-edge of iodine (33.2 keV). The ‘shaping’ process includes selection of kVp and the selection of appropriate X-ray filters. X-ray energies above the K-edge of iodine are absorbed much more strongly than X-ray energies below the k-edge of iodine. Subtraction of the ‘low-energy’ from the ‘high-energy’ image enhances the iodine contrast while suppressing the anatomical ‘clutter’ of normal breast anatomy, which increases the conspicuity of breast cancer. An advantage of dual-energy contrast-enhanced mammography is that both images are obtained sequentially with very short inter-exposure time; therefore, the subtracted image is not degraded by breast movement or blood vessel pulsation. This is not the case with temporal subtraction imaging, since typically there may be a delay of up to a minute separating image acquisition.

Other authors (5,6) have published on the use of molybdenum disulfide (MoS₂) as an alternative to iodinated contrast agents. However, the specific goals of these authors were to examine MoS₂ as a potential general X-ray contrast agent with lower nephrotoxicity than current iodinated contrast agents, to examine its X-ray absorption properties compared to iodine, and to investigate its ability to accumulate in tumor. With regard to the efficacy of Mo as an X-ray absorber, these authors compared the CT densities for scanned solutions of Mo and I and showed that when normalized to the same molecular mass, iodine required 9.8 times the mass of Mo to produce equal CT density in Hounsfield Units.

An important quality of a new mammographic imaging system would be to obtain the highest quality images, such that the number of false-positives and false-negatives in detecting breast cancer would be minimized. Therefore, a need exists for a system and method for acquiring X-ray images to alleviate issues associated with specificity and sensitivity in current designs.

### SUMMARY OF THE INVENTION

Disclosed is a novel technique of breast imaging—‘contrast-enhanced energy-subtraction mammography’ or ‘CEESM’—that employs a molybdenum-based contrast agent. The principle of CEESM is to utilize the K-edge of an element, such as molybdenum, to enable a dual-energy mammographic exposure to be made using existing rhodium and molybdenum X-ray spectra. CEESM with molybdenum-based contrast agents can significantly exceed the diagnostic sensitivity of conventional mammography (typically 60-70%) and approaches the much higher diagnostic sensitivity of contrast-enhanced breast MM (typically 80-90%). Furthermore, the utilization cost of CEESM would not be significantly higher than that for conventional mammography and, therefore, would permit its widespread introduction for improved screening. This would make a major impact on public health related to the early detection of breast cancer.

### BRIEF DESCRIPTION OF THE DRAWINGS

FIG. 1 illustrates the tabulation of the sensitivity and specificity of conventional X-ray mammography and contrast-enhanced breast MM.

FIG. 2 is a diagrammatic representation of the X-ray spectra produced by an X-ray tube with separate molybdenum and rhodium target materials, and the X-ray absorption coefficient variation with X-ray energy for molybdenum.

FIG. 3 illustrates a fluorescence image of a mouse bearing 100 mm³ S-180 tumor 30 minutes post-injection of 0.12 mg/g of MoS₂-PEG (molybdenum-disulfide-polyethyleneglycol) (5).

FIG. 4 tabulates the elements in the periodic table ordered according to increasing atomic number (Z) and K-edge energy in keV.

FIG. 5 represents a diagram of the image subtraction principle.

FIG. 6 illustrates a diagrammatic representation of the mathematical model used to validate theoretically the present invention.

FIG. 7 represents the mathematical equations used to calculate contrast and percent-coefficient-of-variation generated by the model shown in FIG. 6.

FIG. 8A illustrates contrast vs. Rh filter thickness, for Mo, Rh, and CEESM mammograms.

FIG. 8B illustrates contrast vs. Mo filter thickness, for Mo, Rh, and CEESM mammograms.

FIG. 8C illustrates contrast vs. Mo concentration in breast tumor, for Mo, Rh, and CEESM mammograms.

FIG. 8D illustrates contrast vs. Mo Tumor/Blood ratio, for Mo, Rh, and CEESM mammograms.

FIG. 8E illustrates contrast vs. compressed breast thickness, for Mo, Rh, and CEESM mammograms.

FIG. 8F illustrates contrast vs. kVp, for Mo, Rh, and CEESM mammograms.

FIG. 8G illustrates percent-coefficient-of-variation vs. compressed breast thickness, for Mo, Rh, and CEESM mammograms.

FIG. 8H illustrates percent-coefficient-of-variation vs. Air KERMA, for Mo, Rh, and CEESM.

FIG. 9 represents an aluminum ‘Rose’ phantom, designed to provide experimental validation of the CEESM technique.

FIG. 10 represents experimentally obtained Mo, Rh, and CEESM X-ray images of the Rose phantom.

FIG. 11A is a graphical quantitative comparison of relative contrast values for iodine and molybdenum contrast agents.

FIG. 11B is a tabulated quantitative comparison of relative contrast values for iodine and molybdenum contrast agents.

### DETAILED DESCRIPTION OF THE INVENTION

Conventional mammography is currently performed with single-energy X-ray spectra using either rhodium (Rh), molybdenum (Mo), or tungsten (W) X-ray spectra. The Rh and Mo X-ray spectra are produced by Rh and Mo anodes in so-called ‘dual-energy’ X-ray tubes. Such X-ray tubes were originally designed for screen-film mammography (i.e., either Mo or Rh X-rays would be used depending on breast size and composition), but are still used nowadays for digital mammography, although not too frequently. Since the advent of digital mammography around 2000 and breast tomosynthesis (DBT) since 2003, single-energy X-ray tubes producing tungsten target X-rays filtered by a variety of metals (e.g., Al, Ag, Rh) are more frequently used.

Theoretical studies in the late 1970's and early 1980's showed that in order to maximize the contrast of mammographic X-ray imaging using screen/film technology, while concurrently minimizing radiation dose to the breast, monoenergetic X-rays of 17-20 keV energy were ideally required (13). In the early 1990's, it was demonstrated experimentally that an X-ray spectrum produced by a Mo anode combined with a Mo filter complied well with these optimized theoretical requirements (14). However, for large and/or dense breasts, the penetration of the Mo X-ray spectra was found to be inadequate, resulting in significantly elevated breast dose. To remediate this situation, the element rhodium (Rh) was introduced in the early 1990's as an alternative X-ray target material to molybdenum (15). Although the energy of the characteristic X-ray spectrum of Rh is only about 3 keV higher than that of Mo, it proved sufficient to provide the increased X-ray penetration required for larger or denser breasts, resulting in almost the same image quality as for Mo X-rays, but with typically 30-50% lower dose to the breast.

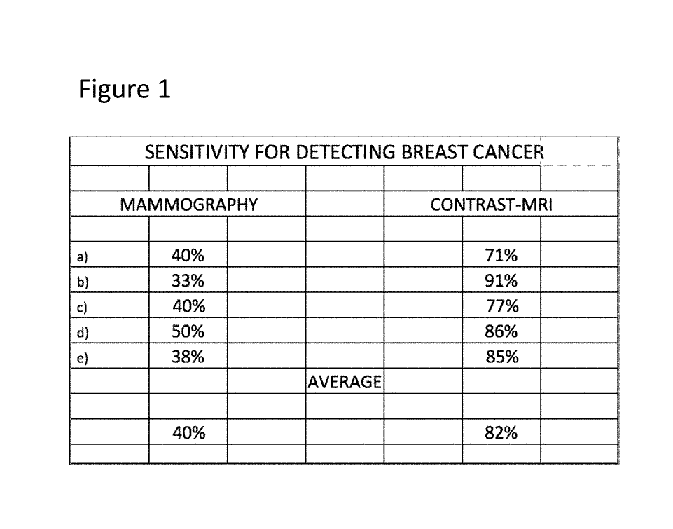

The present invention relates to ‘contrast enhanced energy subtraction mammography’ (CEESM), using a molybdenum-based contrast agent to improve the sensitivity of X-ray mammography from an average of 40% reported for mammography to an average of 82% reported for contrast-enhanced MRI. This comparison is summarized in FIG. 1, which lists the results of five clinical studies referenced in (9). A very large improvement in diagnostic accuracy attainable with breast MRI (80-90%) compared to conventional X-ray mammography (60-70%).

Specifically the invention exploits a combination use of a molybdenum-based injectable contrast agent, combined with Rh and Mo dual-energy X-ray exposures and K-edge image subtraction. In some embodiments, the molybdenum-based contrast agent is molybdenum-disulfide-PEG (MoS₂-PEG), an injectable contrast agent, wherein ‘PEG’ is a radiologically inert solubilizing agent for the MoS₂. Following K-edge subtraction of the Mo and Rh generated X-ray images, the resulting CEESM image provides substantially more MoS₂ contrast compared to current iodinated contrast agents such as Iohexole.

Additionally, the MoS₂ contrast agent that the present invention piggybacks on possesses an additional potential benefit for general X-ray use due to its anticipated lower nephrotoxicity compared to current iodinated contrast agents (16).

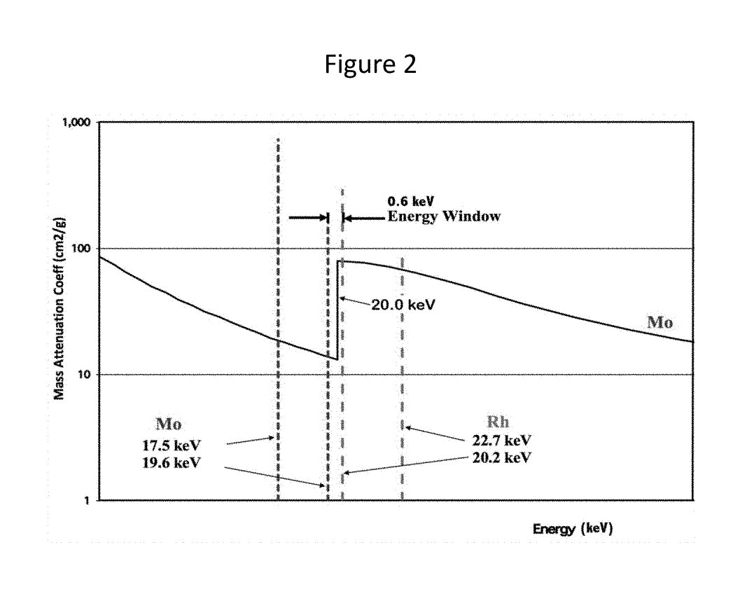

The CEESM method described herein depends on a combination of the absorption properties of Mo and Rh generated X-ray spectra and the K-edge feature of the Mo in the Mo-based contrast agent. The most dominant features of the Mo and Rh X-ray spectra, as shown in FIG. 2, are the narrow X-ray peaks (depicted in this conceptual diagram as dashed vertical lines) called ‘characteristic X-rays’. These are conventionally described as the ‘Kα’ and ‘Kβ’ characteristic X-rays, two for Mo and two for Rh, and can be seen in FIG. 2 as four vertical lines. For Mo, the Kα X-ray occurs at 17.5 keV and the Kβ X-ray occurs at 19.6 keV, while for Rh, the Kα X-ray occurs at 20.2 keV and the Kβ X-ray occurs at 22.7 keV.

Of central importance to the present invention is the difference in energy between the upper 19.6 keV Kβ characteristic X-ray of Mo and the lower 20.2 keV Kα characteristic X-ray of Rh, which differ by only 0.6 keV.

### Choice of Contrast Element for the Present Invention

All elements have X-ray absorption characteristics that vary with energy. Typically, as X-ray energy increases, the X-ray absorption of any element decreases until it reaches a critical point at which its absorption curve abruptly rises by about a factor of 6. Thereafter, with further increase in X-ray energy, the absorption curve again decreases. The X-ray energy at which this abrupt rise in absorption occurs is called the ‘K-edge’ of the absorbing element, and represents the condition where the incoming X-ray energy exactly equals the binding energy of the absorbing element's K-shell electrons.

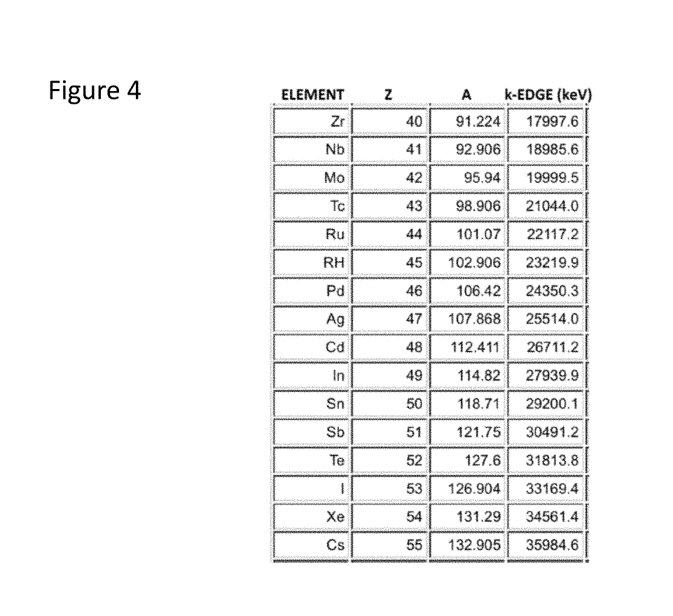

FIG. 4 shows a list of elements from the periodic table, together with their atomic numbers and K-edge energies. The illustration shows that molybdenum has a K-edge energy at 20.0 keV. This is the only element in the periodic table whose K-edge energy exactly ‘fits’ into the narrow 0.6 keV energy gap shown in FIG. 2.

If one assumes a tumor in the breast containing some concentration of Mo, the X-ray absorption coefficient for the two characteristic X-rays of Mo that are just below the K-edge of the Mo in the breast is about 6 times lower than the X-ray absorption coefficient for the two characteristic X-rays of Rh that are just above the K-edge of Mo in the breast. For a 1 cm-thick (as defined along the X-ray beam's axis) breast tumor containing 3 mg/g of Mo, the characteristic X-rays of Rh passing through this imaginary tumor are absorbed about 20 times more than the characteristic X-rays of Mo. Therefore, if the tissue of diagnostic interest is a breast tumor containing, for example, 3 mg/g of Mo, the sensitivity for detecting the tumor can be greatly enhanced by subtracting the image of the breast produced by the Rh X-rays from the image of the breast produced by the Mo X-rays. This K-edge enhanced dual-energy image subtraction principle is the basis of CEESM.

### Tumor Selectivity and Toxicity of Mo Compounds as Required for CEESM

Two important questions need to be addressed if a Mo-based contrast agent is to be considered as a viable new contrast agent: 1) whether the administered Mo-based contrast agent selectively accumulates in breast tumors; and 2) whether the Mo-based contrast agent, at the concentrations needed for successful X-ray imaging, is adequately non-toxic for human use.

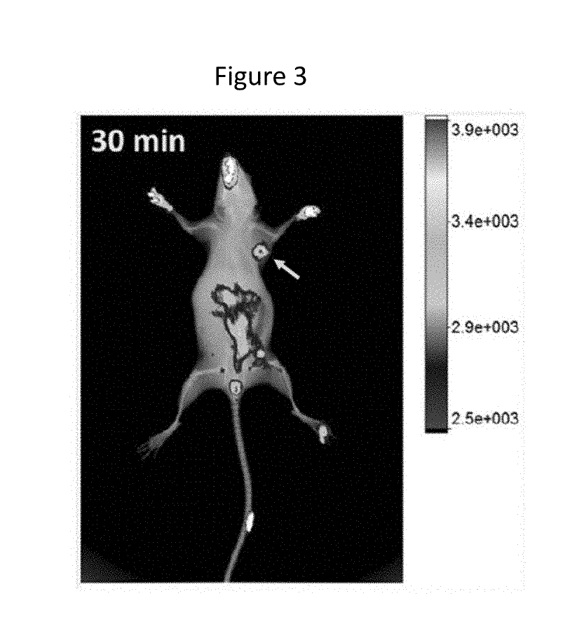

To answer the first question, FIG. 3 shows a fluorescence image of a BALB/c mouse carrying a small 100 mm³ implanted S-180 tumor in the right shoulder, following intraperitoneal injection of 0.12 mg/g of Mo in the form of MoS₂-PEG (10,11). 30 min following MoS₂-PEG injection, the small tumor has taken up sufficient MoS₂-PEG to render it clearly visible (shown by the white arrow). To our knowledge, this is the first, and currently the only, reported selective accumulation of MoS₂-PEG in tumor in vivo.

To answer the second question, reports (10,11) also showed that the intravenous injection of 0.12 mg/g of MoS₂-PEG produced no observable or pathological sequalae in the six mice that comprised the experiment. Furthermore, in toxicity studies on rats and guinea pigs, MoS₂ was found to be ‘non-fatal’ at administered dose levels which, extrapolated to 70 kg human beings, corresponds to 6 mg of Mo/g (17).

CEESM differs in a fundamental way from conventional ‘temporal’ energy-subtraction contrast-enhanced techniques that have been used experimentally for X-ray mammography, as well as since circa 1900 for X-ray angiographic procedures. In these techniques, X-ray images of the breast (or other organ) are taken prior to and following the injection of an iodinated contrast agent, and then subtracted from each other. This requires a mandatory time interval between the two X-ray exposures of many tens of seconds, since the iodinated contrast agent must have sufficient time to reach the breast's (or other organ's) vascular bed and distribute itself between blood, normal tissue, and tumor. This long time-interval is potentially problematic for two reasons: 1) organ motion and blood vessel pulsation can result in incomplete suppression of the normal tissue background anatomy in the subtracted image, thereby reducing the contrast and visual discernibility of tumors; and 2) temporal subtraction cannot be applied to digital breast tomosynthesis, which would eliminate a large fraction of the population that might otherwise benefit from DBT combined with contrast enhanced imaging.

In distinction to conventional iodinated contrast temporal image subtraction techniques, the CEESM technique ‘waits’ for the many tens of seconds that is required for the contrast agent to be selectively absorbed by tumor, and only then obtains two dual-energy exposures in very rapid succession (typically 10-100 milliseconds apart) from which the CEESM subtracted X-ray image is derived. Due to this very short inter-exposure time interval, the probability of breast movement and/or blood vessel pulsation artifacts is extremely low. In addition, the very short inter-exposure time interval would enable the CEESM technique to be applied to digital breast tomosynthesis and would also provide clearer images of the breast and/or tumor vasculature than temporal image subtraction, thereby potentially providing an additional capability for diagnosing breast cancer.

### Mathematical Formulation of the CEESM Technique

The dual-energy subtraction principle as implemented in the CEESM technique can be formulated mathematically as follows.

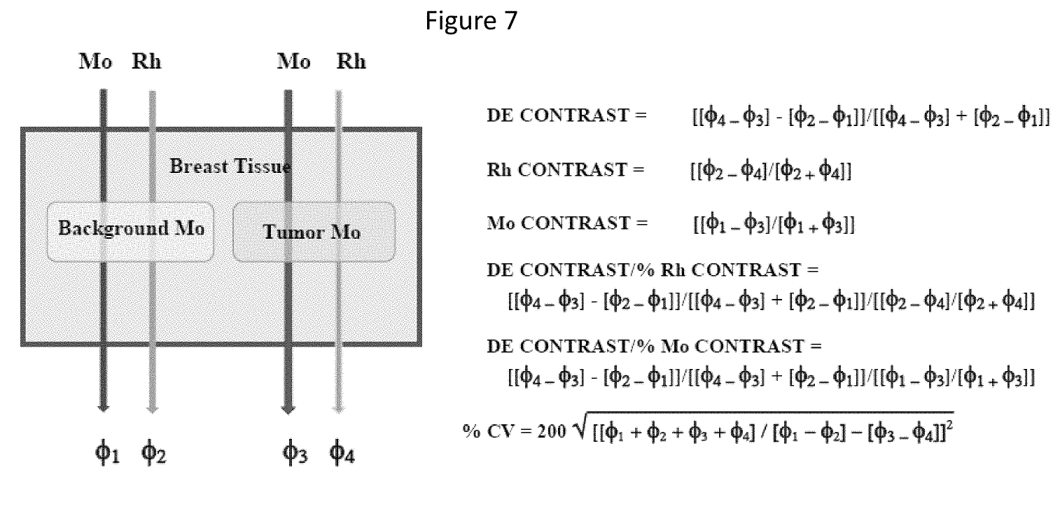

When Mo X-rays produce an image of Mo-containing tumor inside the breast, there are four distinct signal components produced by each detector element; these are: 1) a signal corresponding to Mo X-rays passing through Mo inside the tumor; 2) a signal corresponding to Mo X-rays passing through the tumor with no Mo present; 3) a signal corresponding to Rh X-rays passing through Mo inside the tumor; and 4) a signal corresponding to Rh X-rays passing through the tumor with no Mo present. Signals 2) and 4) will be almost equal, since as previously described, the characteristic X-rays of Rh are only 0.6 keV higher than the characteristic X-rays of Mo, which would result in virtually no difference in X-ray absorption by Mo-free breast tissue. Moreover, as described earlier, the K-edge of the Mo in a 1 cm-thick breast tumor absorbs about 20 times more Rh X-rays than Mo X-rays. Therefore, the Mo X-ray signal will be about 20 times larger than the Rh X-ray signal, irrespective of the Mo concentration within the tumor. (FIG. 7)

One final assumption in this calculation is that all X-rays striking a detector element will be absorbed with equal efficiency.

Based on the above definitions of ϕ₁, ϕ₂, ϕ₃, and ϕ₄,

SIGNAL [Mo]=φ₁+ϕ₂ (1)

SIGNAL [Rh]=ϕ₃+ϕ₄ (2)

Now since the Rh and Mo X-ray signals for the breast with and without Mo present will be almost equal, in equation (1) we can substitute ϕ₃ or ϕ₂, giving,

SIGNAL [Mo]˜ϕ₁+ϕ₃ (3)

Also, since the Mo signal is about 20 times larger than the Rh signal, irrespective of the Mo concentration in the tumor,

ϕ₁˜20*ϕ₄ (4)

Substituting equation (4) for ϕ₁ in equation (3),

SIGNAL [Mo]˜20*ϕ₄+ϕ₃ (5)

Finally, subtracting the Rh signal from the Mo signal yields,

$$\begin{matrix}SIGNAL [ Mo ] - SIGNAL [ Rh ] = [ {20}^{*} {φ}_{4} + {φ}_{3} ] - [ {φ}_{3} + {φ}_{4} ] \\ = 19 \cdot {φ}_{4}\end{matrix} \qquad ( 6 )$$

The left-hand side of equation (6) is the difference signal derived from each individual detector in the detector array following Mo and Rh X-ray exposure subtraction, which we will call the ‘subtraction signal’. The right-hand-side of equation (6) contains no signals associated with Mo-free breast tissue or breast tumor. Therefore, equation (6) shows that the CEESM technique can achieve complete suppression of background breast tissue ‘clutter’ without any significant reduction in the Mo signal due to Mo present in those tissues.

### A Visual Aid to Clarify the Principle of Image Subtraction

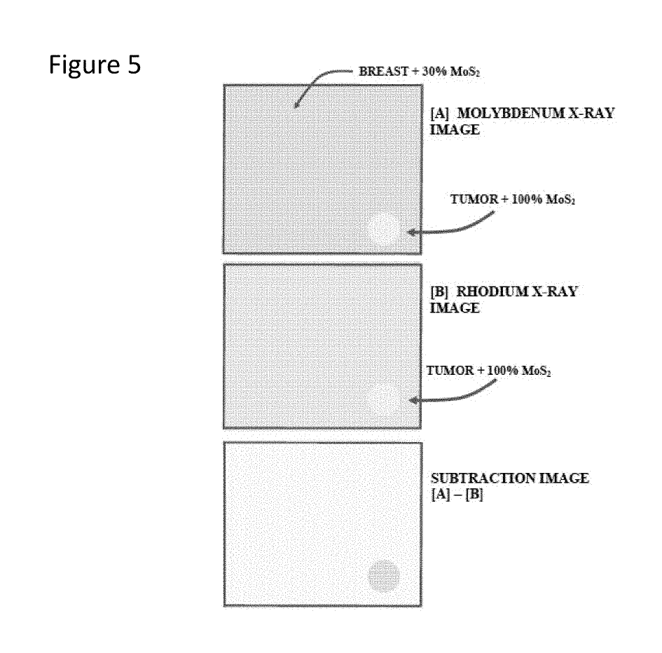

The concept of the CEESM K-edge image subtraction technique as described above may be tricky to follow. In FIG. 5, the X-ray image at the top represents a mammogram of a breast containing a spherical tumor (located in the bottom right-hand corner), containing some quantity of contrast agent—depicted as ‘100%’, obtained with a Rh X-ray exposure. On a relative basis, the normal breast tissue or blood is assumed to contain 30% of the tumor contrast agent (i.e., the tumor/blood ratio is 3:1). In this image, the tumor is poorly visualized due to the low amount of contrast agent it has absorbed. The middle image is obtained with a Mo X-ray exposure, and again the tumor is poorly visualized due to the low amount of contrast agent it has absorbed. However, subtracting these two images from one another produces the bottom image, in which the subtraction process has suppressed the anatomical ‘clutter’ of the normal breast tissue and the contrast agent contained therein, and has significantly enhanced the visibility of the tumor.

### Why the CEESM Technique Requires Mo as a Contrast Agent

The CEESM technique employing Rh and Mo X-rays requires Mo as a contrast agent because the technique only works if the K-edge of the contrast element fits into the narrow 0.6 eV energy window shown in FIG. 2. Mo, with its K-edge energy of 20.0 keV, is the only element in the periodic table that fulfills this requirement.

Why Mo-Based Contrast Agents May be Safer than Iodinated Contrast Agents

There is an additional reason why Mo would be a useful contrast agent for both mammography as well as general X-ray imaging. At the concentrations used clinically, conventional iodinated contrast agents, such as Iohexole, can produce kidney damage in certain vulnerable sub-groups of patients that have compromised kidney function. In patients with normal kidney function, the risk of nephrotoxicity has been reported as ‘less than 1-2%’, while in ‘high-risk’ patients, nephrotoxicity can reach an incidence of 40% (16).

Toxicity of MoS₂ has been tested in rodents. The extrapolated equivalent of 420 g of Mo as MoS₂ in a 70 kg human was reported to be non-fatal in rodents (17). This translates in humans to an average Mo tissue concentration of 6 mg/g. Combined with its fairly high atomic number of 42, Mo as MoS₂ has been proposed as a new, potentially safer, X-ray contrast agent than current iodinated contrast agents. In reference (7) when BALB/c 18-20 g mice bearing a 100 mm³ S-180 tumor in the right shoulder were a) injected intravenously with 240 mg/mL of Iohexole (0.1 mL), and b) injected with 8 mg/mL of MoS₂-PEG (0.1 mL), despite the ˜30-fold lower molecular mass of Mo compared to iodine, tumor discernability with MoS₂-PEG was significantly superior to that with Iohexole. This 30-fold difference at least in part explains the significantly lower nephrotoxicity anticipated with MoS₂-PEG.

Mathematical modeling done for the present invention used a nominal concentration of 3 mg/g of Mo within a breast tumor. Depending on the value of the tumor/blood ratio, the corresponding Mo concentration in blood and/or normal breast tissue correlates more with the manifestation of toxicity than the Mo concentration in tumor, and this concentration would be substantially less than 3 mg/g. ‘Parametric variation’ calculations were done covering a range of Mo tumor concentrations from 1 to 10 mg/g, with corresponding blood/normal breast tissue Mo concentrations of 1-10 times lower.

### Instrumentation

A system according to one example includes an X-ray source including one or more X-ray filters, an imaging X-ray detector, and an immobilization mechanism positioned between the X-ray source and the detector for immobilizing an object to be imaged such as a patient's breast. During image acquisition, X-rays of two or more different energy ranges are generated from the X-ray source by varying at least one X-ray source acquisition parameter, including but not limited to the X-ray filters and X-ray kV. The X-rays propagate through the imaged object and are received by the detector. The composition of the imaged object modulates the X-rays through mechanisms such as attenuation, absorption and scatter, resulting in relatively brighter and darker areas in a detected image. The detected image is processed using computer-processing techniques and the resulting images may be stored and/or displayed at a radiologist's workstation.

The system may include a control module for controlling image acquisition, the control module including a user interface permitting a user to select one or more modes of image acquisition and/or image processing. The user interface may comprise a keypad, touchpad, joystick or other input mechanism that interacts with a computer program executing on a computer system coupled to a display. Such a user interface may enable selection of image acquisition mode depending upon the capabilities of the breast-imaging device.

### Image Acquisition

One example is an X-ray image acquisition system that is optimized for mammography and breast tomosynthesis and is further modified for dual-energy imaging and for the use of a contrast agent. One system that can serve illustratively as a basis for further modifications is the Selenia® Dimensions® tomosynthesis imaging system, manufactured and sold by Hologic, Inc., of Bedford Mass.

This system is a ‘combo-mode’ system capable of acquiring images in either or both 2D and 3D modes, but it should be clear that this is not the only example of a suitable system, and that 2D mammography-only systems also may serve as a basis for modification. Accordingly, the systems and methods described in this patent specification are not limited to a particular starting system that can be used or modified to carry out the required processes.

### Image Acquisition Process Selection

One important characteristic of any digital imaging system is the ability to vary the amount and intensity of radiation used to generate any image. Radiation intensity is related to the atomic number (Z) of the X-ray target, the X-ray current (mA), X-ray Voltage and X-ray beam filtration. Radiation intensity is varied to improve image quality, which in turn can improve diagnostic sensitivity. When radiation intensity increases, quantum mottle (image noise caused by photon absorption) tends to decrease and vice versa.

Many mammography and tomosynthesis systems allow the operator to control X-ray exposure by manually setting technique factors such as mA and mSec. Some systems include an Automatic Exposure Control (AEC) functionality which controls a duration of administration of radiation, turning off the X-ray source when the desired dose has been administered. Automatic Exposure Control (AEC) methods may vary the dosing parameters, including exposure time, kV, mA and filter modes for an image to vary the exposure and the radiation intensity.

A breast imaging system according to examples described in this patent specification combines the capabilities of combined 2D and/or 3D breast X-ray imaging with benefits from contrast image acquisition processes. Biopsy capability (Stereotactic or tomosynthesis guided) may be integrated into the system, with lesion localization software utilizing any images selected from a group including simple 2D images, 3D projection images, 3D reconstructed data, or any of the 2D, 3D projection and 3D reconstructed data obtained during a dual energy or background subtraction image acquisition process.

With such arrangements, the following image protocols are supported: Contrast imaging (background subtraction) using a single high or low energy image acquisition technique, in 2D or 3D mode; Dual-energy contrast imaging in 2D or 3D mode; Dual-energy contrast imaging in 3D mode, wherein high and low energy exposures occur at different angles during a tomosynthesis scan; high and low energies can be reconstructed separately and combined to form the dual energy volume;

Dual-energy imaging in a combo system that acquires dual-energy 2D and dual-energy 3D images; in combo imaging mode, where the 2D image data set is acquired using a single energy, and the 3D image data set is acquired using dual-energy imaging; in combo imaging mode, where the 2D image data set is acquired using dual-energy imaging, and the 3D image data set is acquired using a single energy image; Tomosynthesis imaging mode, wherein among a total of N views in a contrast tomo scan wherein the breast remains in compression throughout the scan, different projection images are allotted different dose, kVps, mAs and filters for greater flexibility of different applications; Tomosynthesis mode wherein a low energy scans and high energy scans alternate in a series of acquired projection images; Tomosynthesis mode wherein low energy and high energy scans are performed for the projection images, in unequal ratios in user selectable patterns; stereotactic biopsy using contrast agent, and either dual energy or background subtraction imaging; Upright biopsy using tomosynthesis scan images obtained using a contrast agent and either dual-energy or back ground subtraction imaging.

Other variations of combinations of contrast imaging and image acquisition modes are within the scope of this patent specification.

### Image Acquisition Parameter Selection

Once an image acquisition mode and an acquisition process are identified, acquisition parameters and image processing techniques can be varied at a projection image granularity by varying at least one of kV, mA and/or filter for each 2D image capture. Several modifications to existing mammography and/or tomosynthesis breast imaging systems may be made to support contrast imaging. For example, within the X-ray source, mechanisms that allow fast Switching between kV, mA and X-ray beam filters may be provided to support dual-energy imaging between and within image capture modes. For example, an X-ray filter wheel may be provided to switch filters between low and high energy pulses. A variety of different filters, such as rhodium, silver, aluminum, copper and cesium iodide may be provided to provide the desired energy for different contrast agents.

### Image Processing Selection

The new system also allows different image processing to be performed on received images, where the image processing techniques may be determined in response to a type of acquisition (i.e., a tomosynthesis acquisition, a 2D acquisition, a dual-energy acquisition, a contrast acquisition). Thus, for example, images acquired using high energy may be processed using different algorithms than images acquired using low energy. The image processing technique may be preprogrammed based on the selected acquisition mode or alternatively may be selected in response to user input. For the purposes of this patent specification, image processing refers to any manipulation and combination of the images, including noise filtering and image reconstruction. Some of the processing may be a function of the acquisition mode. For example, when performing background subtraction contrast imaging using tomosynthesis images, pre- and post-injection projection images may be subtracted, and the resulting signal shifted to register the images to compensate for patient motion.

In one embodiment, the system enables the utilization of either gain controlled images or air-map corrected images as a basis for the contrast image processes (i.e., the images may be processed prior to the subtraction or addition processes). Gain controlled images are images that have been processed to compensate for system gain to increase SNR, for example using techniques described in U.S. Pat. No. 7,991,106 (incorporated by reference).

### Display

A display of the new system may be used to display images captured using any of the modalities (2D, 3D, combo), using any image acquisition process. The display includes the ability to display the images in a variety of configurations, including singularly, side by side, toggled, or in cine-mode. With Such an arrangement, a health professional may simultaneously view (or toggle between, or view in cine), the 2D image, 3D projection image or 3D Slice image of a breast, at either the low energy acquisition, high energy acquisition, or following subtraction of the two, with or without the use of contrast agents, thereby enhancing the ability to visualize and characterize lesions.

Although the above has described the use of the system with regard to acquisition of both tomosynthesis and mammogram images, this patent specification is not limited to an integrated multi-mode system but applies to any system that is capable of performing tomosynthesis.

### Methods of the Invention

In some embodiments, the invention relates to a method of dual energy X-ray imaging, comprising the steps of:

administering to a subject an effective amount of a molybdenum-based contrast agent; wherein said administration is intravenous;

acquiring a first image of a tissue of the subject with a low energy spectrum; and

acquiring a second image of the tissue of the subject with a high energy spectrum.

In some embodiments, the low energy spectrum comprises a low energy spectrum filtered with a molybdenum filter. In some embodiments, the high energy spectrum comprises a high energy spectrum filtered with a rhodium filter. In some embodiments, the molybdenum filer is a 0.01 mm Mo filter.

In some embodiments, the low energy spectrum and high energy spectrum are both acquired within less than 100 milliseconds elapsed time. In some embodiments, the low energy spectrum and high energy spectrum are both acquired within less than 90 milliseconds elapsed time. In some embodiments, the low energy spectrum and high energy spectrum are both acquired within less than 80 milliseconds elapsed time. In some embodiments, the low energy spectrum and high energy spectrum are both acquired within less than 70 milliseconds elapsed time. In some embodiments, the low energy spectrum and high energy spectrum are both acquired within less than 60 milliseconds elapsed time. In some embodiments, the low energy spectrum and high energy spectrum are both acquired within less than 50 milliseconds elapsed time. In some embodiments, the low energy spectrum and high energy spectrum are both acquired within less than 40 milliseconds elapsed time. In some embodiments, the low energy spectrum and high energy spectrum are both acquired within less than 30 milliseconds elapsed time. In some embodiments, the low energy spectrum and high energy spectrum are both acquired within less than 20 milliseconds elapsed time. In some embodiments, the low energy spectrum and high energy spectrum are both acquired within less than 10 milliseconds elapsed time. In some embodiments, the low energy spectrum and high energy spectrum are both acquired within less than 5 milliseconds elapsed time. In some embodiments, the low energy spectrum and high energy spectrum are both acquired within less than 1 milliseconds elapsed time.

One aspect of the invention is a method of multi-mode X-ray examination of a contrast-enhanced patient's breast, comprising the steps of:

introducing intravenously a vascular contrast agent into a patient's breast; wherein the contrast agent is a molybdenum-based contrast agent;

obtaining (i) a low-energy X-ray 2D mammogram of the breast, and (ii) a high-energy X-ray 2D mammogram of the breast;

wherein said low-energy and high-energy 2D mammograms are taken in a single breast compression;

computer-processing the 2D low-energy mammogram and the 2D high-energy mammogram to form a weighted combination 2D image highlighting vascularity in the breast; and

displaying the weighted combination 2D image;

wherein the displayed weighted combination 2D image facilitates identification of a position of a possible abnormality in two dimensions.

In some embodiments, the method further comprises obtaining a multiplicity of single-energy X-ray 2D tomosynthesis projection images of the patient's breast; computer-processing the 2D tomosynthesis projection images into 3D slice images that represent respective slices of the breast; and displaying the weighted combination 2D image and one or more of the 3D slice images; wherein the one or more 3D slice images facilitate identification of a position of the abnormality in three dimensions and visualization of the appearance of the abnormality in respective slice images.

In some embodiments, the multiplicity of single-energy X-ray 2D tomosynthesis projection images are acquired prior to acquiring the low-energy and high-energy X-ray 2D mammograms.

In some embodiments, displaying comprises concurrently displaying the combination 2D image and at least one of the 3D slice images. In some embodiments, displaying comprises displaying the combination 2D image and at least one of the 3D slice images in mutual registration. In some embodiments, displaying comprises concurrently displaying the combination 2D image and a plurality of the 3D slice images. In some embodiments, displaying comprises concurrently displaying the combination 2D image and a subset of the 3D slice images, wherein the subset comprises 3D slice images in which the abnormalities appear and the subset is computationally selected in response to an identification of the abnormality in the combination 2D image. In some embodiments, the abnormalities appear on the tomosynthesis image, but not on the combination 2D image.

Another aspect of the invention related to a method of multi-mode X-ray examination of a contrast-enhanced patient's breast, comprising the steps of:

introducing intravenously a vascular contrast agent into a patient's breast; wherein the contrast agent is a molybdenum-based contrast agent;

obtaining (i) a multiplicity of single-energy X-ray 2D tomosynthesis projection images of the breast, (ii) a low-energy X-ray 2D mammogram of the breast, and (iii) a high-energy X-ray 2D mammogram of the breast; wherein said 2D tomosynthesis projection images are obtained before said 2D mammograms, and the 2D tomosynthesis projection images and the mammograms are taken in a single breast compression;

computer-processing the 2D low-energy mammogram and the 2D high-energy mammogram to form a weighted combination 2D image highlighting vascularity in the breast; and
displaying the weighted combination 2D image and one or more of the 3D slice images; wherein the displayed weighted combination 2D image facilitates identification of a position of a possible abnormality in two dimensions, and the displayed one or more 3D slice images facilitate identification of the position of the abnormality in three dimensions and visualization of appearance of the abnormalities in respective slice images.

In some embodiments, the method further comprises obtaining 3D slice images of the patient's breast that represent respective slices of the breast and are reconstructed through computer-processing a multiplicity of single-energy X-ray 2D tomosynthesis projection images.

In some embodiments, the 3D slice images of the patient's breast are obtained prior to acquiring the low-energy and high-energy X-ray 2D mammograms.

In some embodiments, displaying comprises concurrently displaying the weighted combination 2D image and at least one of the 3D slice images. In some embodiments, displaying comprises displaying the weighted combination 2D image and at least one of the 3D slice images in mutual registration. In some embodiments, comprises concurrently displaying the weighted combination 2D image and a plurality of the 3D slice images.

Another aspect of the invention relates to a method of multi-mode X-ray examination of a patient's breast, comprising the steps of:

obtaining, from a single compression of the breast (i) 3D slice images representing respective slices of a patient's breast formed by computer-processing, through a reconstruction algorithm, from a multiplicity of X-ray 2D tomosynthesis projection images of the breast taken at a plurality of angles of the imaging X-ray beam relative to the breast and (ii) a 2D weighted combination image of a low-energy 2D X-ray mammogram of the breast and a high-energy 2D X-ray mammogram of the breast in the presence of a contrast agent therein, wherein the 2D mammograms are obtained after acquiring the 2D tomosynthesis projection images; and

concurrently displaying on a computer display the 2D weighted combination image and one or more of the 3D slice images; wherein the displayed weighted combination 2D image facilitates identification of a position of a possible abnormality in two dimensions, and the displayed one or more 3D slice images facilitate identification of the position of the abnormality in three dimensions and visualization of appearance of the abnormalities in respective slice images.

In some embodiments, the 3D slice images are obtained prior to the obtaining the 2D combination image. In some embodiments, the 2D tomosynthesis projection images are single-energy images. In some embodiments, the combination 2D image is a weighted combination of the high-energy and low-energy images. In some embodiments, the 2D tomosynthesis projection images are dual-energy images.

In some embodiments of the invention, the possible abnormality is a vascular abnormality.

In some embodiments, the molybdenum-based contrast agent of the invention is molybdenum disulfide. In some embodiments, the molybdenum-based contrast agent is MoS₂-PEG.

In some embodiments, molybdenum-based contrast agent is administered to the subject in an amount sufficient to produce a concentration of about 0.3-3 mg of molybdenum/g; wherein mg of molybdenum/g is mg of molybdenum/g of tissue. In some embodiments, molybdenum-based contrast agent is administered to the subject in an amount sufficient to produce a concentration of about 4 mg of molybdenum/g. In some embodiments, molybdenum-based contrast agent is administered to the subject in an amount sufficient to produce a concentration of about 3 mg of molybdenum/g. In some embodiments, molybdenum-based contrast agent is administered to the subject in an amount sufficient to produce a concentration of about 2 mg of molybdenum/g. In some embodiments, molybdenum-based contrast agent is administered to the subject in an amount sufficient to produce a concentration of about 1 mg of molybdenum/g. In some embodiments, molybdenum-based contrast agent is administered to the subject in an amount sufficient to produce a concentration of about 0.75 mg of molybdenum/g. In some embodiments, molybdenum-based contrast agent is administered to the subject in an amount sufficient to produce a concentration of about 0.5 mg of molybdenum/g. In some embodiments, molybdenum-based contrast agent is administered to the subject in an amount sufficient to produce a concentration of about 0.3 mg of molybdenum/g. In some embodiments, molybdenum-based contrast agent is administered to the subject in an amount sufficient to produce a concentration of about 0.2 mg of molybdenum/g.

### Rose Phantom

Another aspect of the invention relates to an aluminum ‘Rose’ phantom, comprising a plurality of milled wells of varying diameter and depth, wherein each of the plurality of milled wells comprises a Mo-containing compound.

In some embodiments, the Mo-containing compound is a Mo-based paste. In some embodiments, the Mo-based paste is a grinding paste.

In some embodiments, each of the plurality of milled wells independently has a depth of 2, 1, or 0.5 mm. In some embodiments, the milled wells have a depth of 2 mm. In some embodiments, the milled wells have a depth of 1 mm. In some embodiments, the milled wells have a depth of 0.5 mm.

In some embodiments, each of the plurality of milled wells independently has a diameter of 5, 3, or 2 mm. In some embodiments, the milled wells have a diameter of 5 mm. In some embodiments, the milled wells have a diameter of 3 mm. In some embodiments, the milled wells have a diameter of 2 mm.

In some embodiments, each of the plurality of milled wells independently has a depth of 2, 1, or 0.5 mm; and each of the plurality of milled wells independently has a diameter of 5, 3, or 2 mm.

### EXAMPLES

### Example 1: Theoretical Calculations to Validate the CEESM Technique

The mathematical calculations that were done for the present invention conservatively used a nominal concentration of 3 mg/g of Mo within a 1 cm³ breast tumor. Assuming a tumor/blood Mo ratio of 3:1, the Mo concentrations in blood and normal tissues would correspond to about 1 mg/g. In general, it is the Mo concentration in blood and/or normal breast tissue that correlates with the manifestation of toxicity rather than the Mo concentration in tumor, since the tumor volume is extremely small compared to the combined blood and normal tissue volumes. Thus, the nominal MoS₂-PEG concentrations assumed for blood and normal tissues are 4-6-fold lower than the 6 mg/g of Mo concentration referred to earlier that was reported as being non-fatal to rodents.

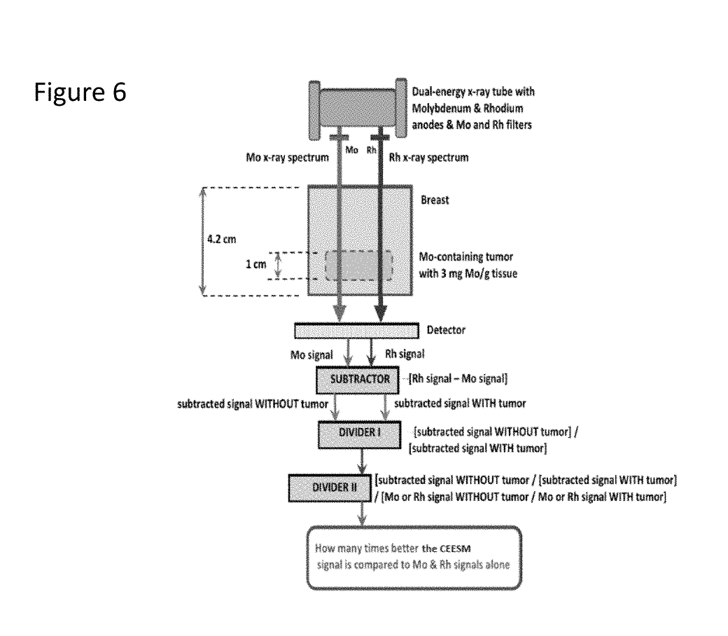

The mathematical model used to theoretically validate the CEESM concept is shown in FIG. 6.

A dual-energy X-ray tube having separate Mo and Rh X-ray targets produces both Mo and Rh X-rays. The X-ray beams are shown by the thick vertical arrows at the top of illustration 6. Each X-ray beam is first filtered by a 0.03 mm Mo filter for the Mo X-ray beam and by a 0.03 mm Rh filter for the Rh X-ray beam (typical filtration values for Rh and Mo X-ray spectra used in mammography). The filtered X-rays then enter a 4.2 cm-thick breast. In mammography, compression devices compress the breast to improve image quality and reduce breast dose, and 4.2 cm is considered by the FDA to be a typical average compressed breast thickness). Within the 4.2 cm breast is a 1 cm-thick (measured along the axis of the X-ray beams) region of Mo-containing tumor. As previously mentioned, the nominal concentration of Mo within this breast tumor was conservatively set as 3 mg/g. Separated by only a few tens of milliseconds, the Mo and Rh X-rays emerge from the breast and strike one of an array of digital detector elements. The Mo and Rh X-rays absorbed in the detector create separate electrical signals, proportional to their respective fluences (‘intensities’, expressed as X-ray photons/cm²). A digital module labeled ‘SUBTRACTOR’ subtracts the Mo signal from the Rh signal and generates a ‘difference’ signal. The Mo-containing tumor in the breast is now replaced by normal breast tissue, a second set of Mo and Rh X-ray exposures is made, and a second ‘difference’ signal is generated. There are now two difference signals: one produced with the Mo-containing tumor present, the second produced with the Mo-containing tumor replaced by normal breast tissue. A digital divider module, labeled ‘DIVIDER-I’, takes the difference signal with the Mo-containing tumor present and divides it by the difference signal with the Mo-containing tumor replaced by normal breast tissue. The resulting ‘CEESM contrast ratio’ defines the contrast with which the Mo-containing tumor in the breast can be discerned against a background of normal breast tissue containing a lower—or zero—concentration of Mo. Finally, a second divider module, labeled ‘DIVIDER-II’, divides the CEESM contrast ratio by the single-energy Mo and Rh contrast ratios, and yields two contrast ‘figures-of-merit’ for the CEESM technique relative to contrast ratios achievable with dual-energy Rh and Mo X-ray exposures.

The mathematical methodology used to analyze the CEESM technique follows what is commonly referred to as ‘recursive analysis’. In this statistical approach, all variable parameters are first set to their ‘nominal’—or typical values. Then, each parameter in turn is varied over a range of settings while the remaining parameters are forced to remain at their nominal settings.

There are eight independent variable parameters in the CEESM mathematical model, as listed below together with their ‘nominal’ values in parentheses.

### Eight Independent Variable Parameters:

1. Mo concentration in tumor (3 mg/g)

2. Mo concentration in normal breast tissue or blood (0.3 mg/g)

3. Mo-based tumor-to-blood ratio (10:1)

4. Kilovoltage (30 kVp)

5. Mo filter thickness (0.03 mm)

6. Rh filter thickness (0.03 mm)

7. Compressed breast thickness (4.2 cm)

8. Air KERMA (1.0 mGy)

Similarly, there are four calculated dependent variables as listed below.

### Four Dependent Variable Parameters:

1. %-contrast for Mo X-ray exposure

2. %-contrast for Rh X-ray exposure

3. %-contrast for the CEESM

4. %-coefficient-of-variation of the contrast values calculated in 1-3

A summary of the equations used to calculate the contrast and coefficients-of-variation factors is shown in FIG. 7.

### Example 2: Results of Theoretical Calculations to Validate the CEESM Technique

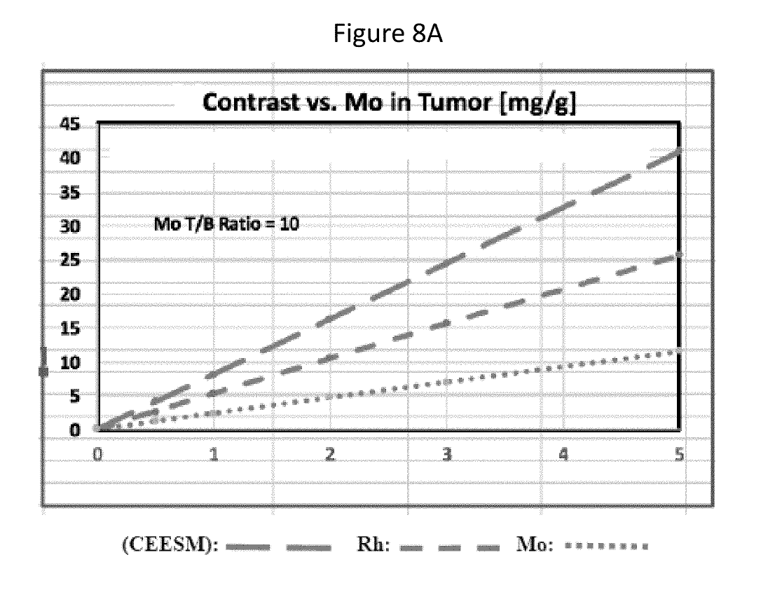

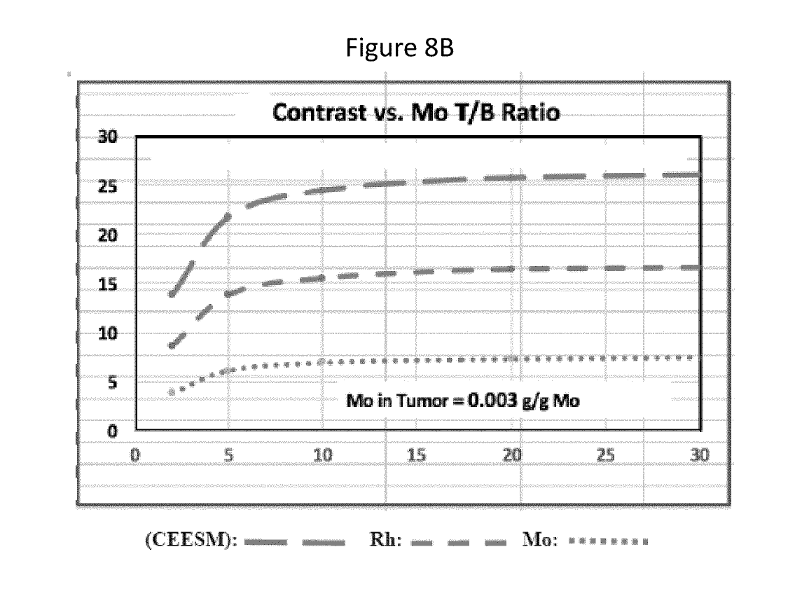

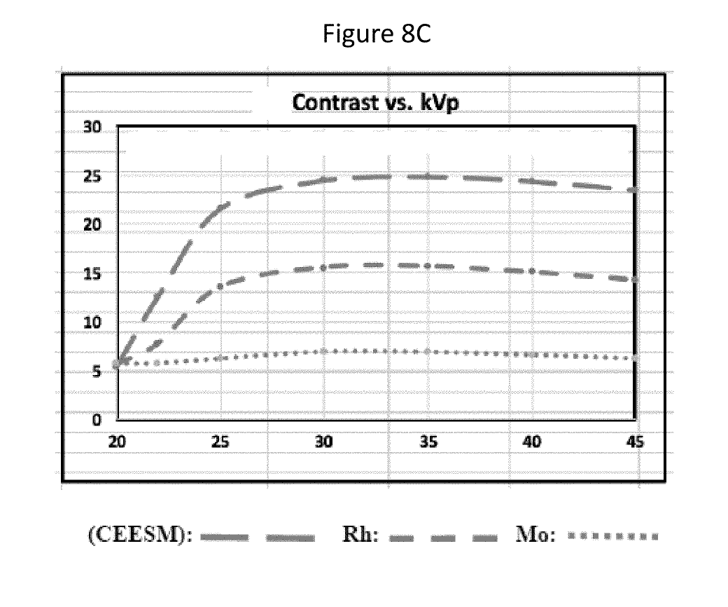

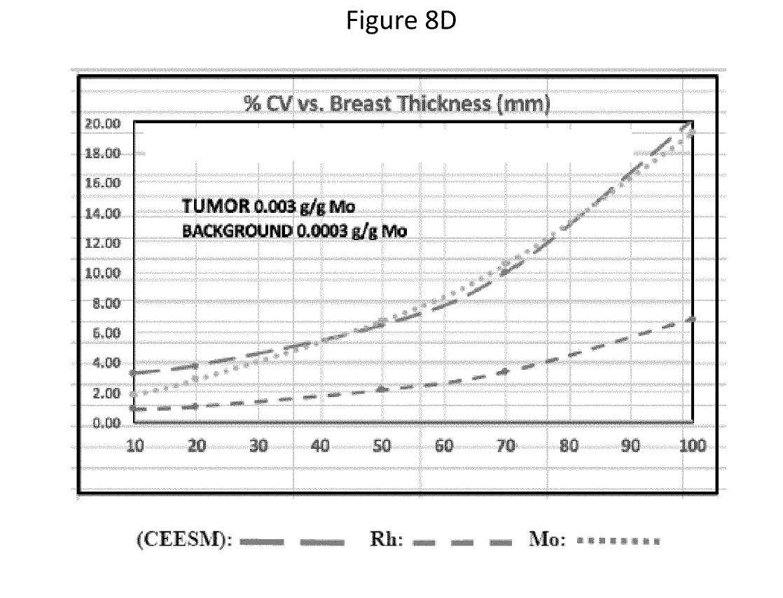

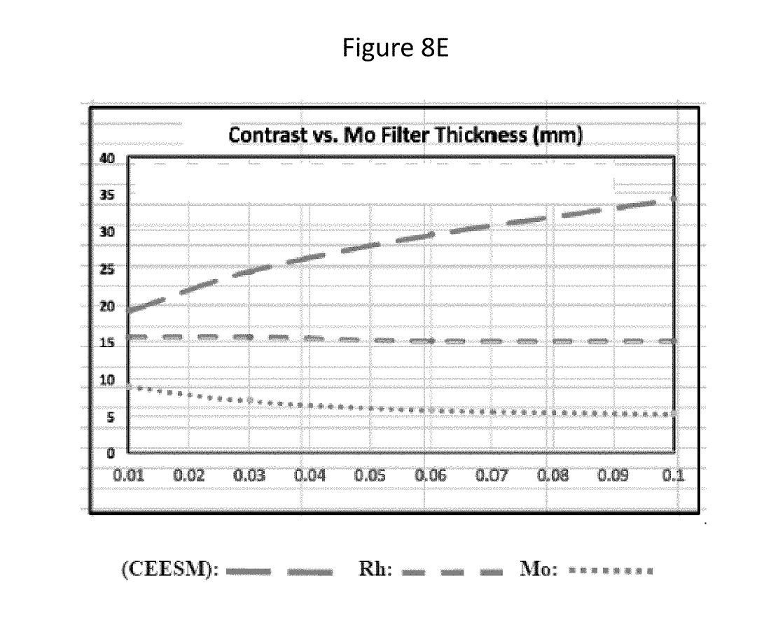

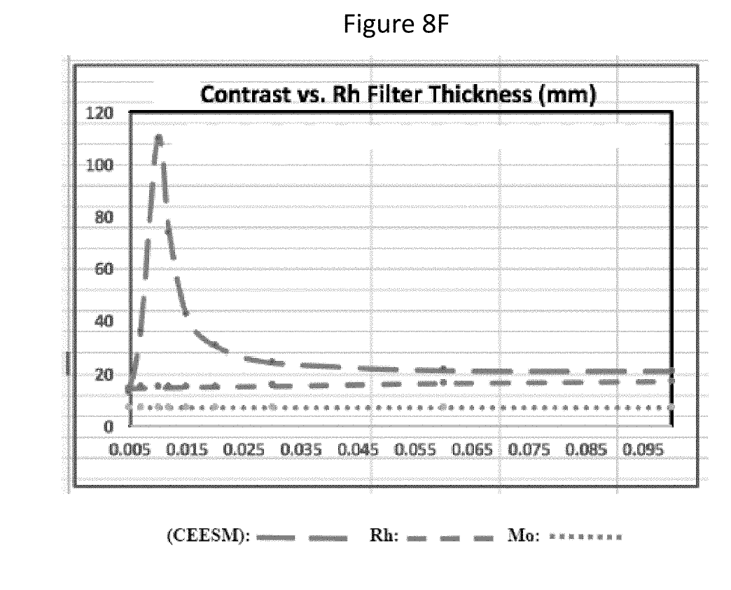

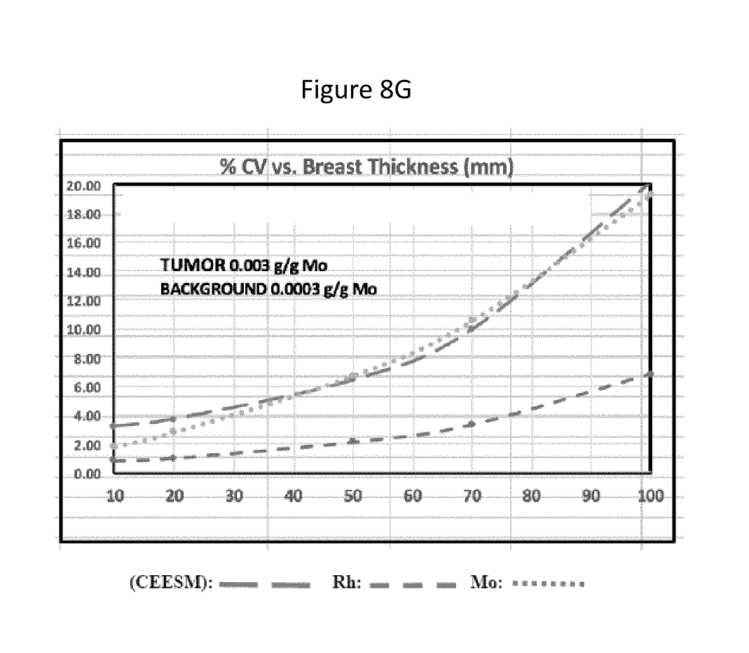

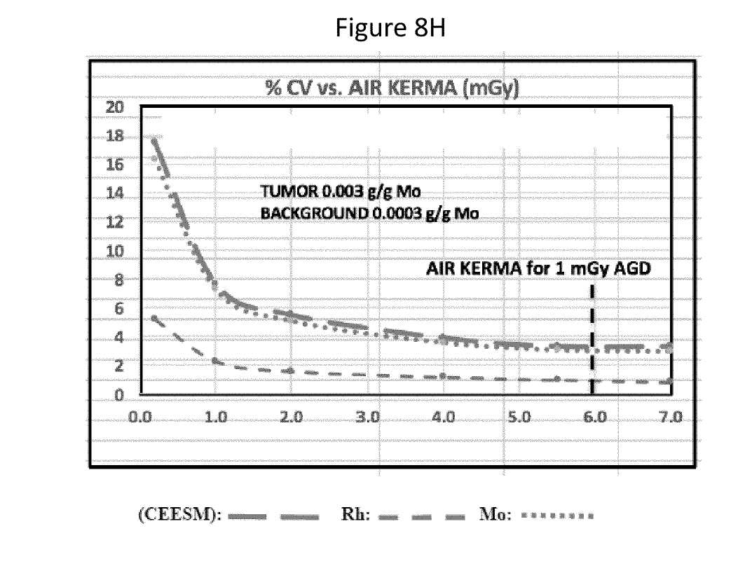

FIG. 8A shows %-contrast vs. Mo concentration in tumor. As would anticipated, the %-contrast for all three curves increases linearly with Mo concentration in tumor, with the CEESM curve being about 1.5× and 3.5× higher than the single-energy Rh and Mo curves at all Mo concentrations.

Realistically, however, one would never expect a contrast agent to locate exclusively in just one anatomical compartment. Illustration 8b models two coexisting Mo concentrations: one in breast tumor and the other in normal tissue, e.g., normal breast tissue or blood. These two Mo concentrations are modeled as a ‘Mo tumor/blood ratio’.

FIG. 8B shows %-contrast vs. Mo tumor/blood ratio, with tumor Mo concentration fixed at 3 mg/g. At tumor/blood ratios of 10 and higher, the curves asymptote, and the CEESM curve becomes 1.5× and 3.5× higher than the single-energy Rh and Mo curves. At tumor/blood ratios lower than 10, all three curves are lower but the ratio for CEESM relative to Rh and Mo is maintained at 1.5× and 3.5×. These results show that the advantage of the CEESM technique over the single-energy techniques vis-à-vis contrast is independent of Mo concentration in tumor or Mo tumor/blood ratio.

FIG. 8C shows %-contrast vs. kVp. From 30-45 kVp, the three %-contrast curves are virtually horizontal, with the CEESM curve again remaining about 1.5× and 3.5× higher than the Rh and Mo curves. The apparent insensitivity of contrast to variation in kVp is one of the unique benefits of using Mo as a contrast agent. With iodinated contrast agents, or in the absence of contrast agent, contrast is inversely related to breast radiation dose, so in choosing kVp one is faced with balancing image quality against radiation dose. With Mo as a contrast agent, however, one can raise the kVp with impunity, and in so doing greatly reduce the radiation dose to the breast. This is a very important collateral advantage of the CEESM technique.

FIG. 8D shows %-contrast vs. compressed breast thickness. For a very large compressed breast of 10 cm, the three %-contrast curves asymptote, with the CEESM curve 1.2× and 2× higher relative to the Rh and Mo curves. For a breast of 5 cm or less, the CEESM %-contrast curve is 2.2× and 4.5× times higher than the Rh and Mo curves. This shows that the CEESM technique results in a significant contrast advantage over the single-energy Mo and Rh techniques, with a greater advantage for the smaller breast (or larger but more-fatty breast.)

FIG. 8E shows %-contrast vs. Mo filter thickness. As would be expected, variations in Mo filter thickness produce minimal effects on single-energy Rh contrast. However, as Mo filter thickness increases, CEESM contrast increases substantially while the Mo curve gradually decreases. With the frequently used Mo filter thickness of 0.03 mm, the CEESM contrast is about 1.7× and 3.3× higher than single-energy Rh and Mo contrast.

FIG. 8F shows %-contrast vs. Rh filter thickness. Increasing Rh filter thickness beyond the customary values of about 0.03 mm, all three contrast curves asymptote, but decreasing the Rh filter thickness down to 0.01 mm causes the CEESM curve to peak at 7× and 16× higher than the single-energy Rh and Mo curves. This ‘singularity’ in the CEESM contrast response suggests that Rh filter thicknesses of significantly less than the customary 0.03 mm would be desirable to maximize CEESM efficacy. However, such a maneuver would also increase average glandular dose (AGD).

FIG. 8G shows %-CV vs. breast thickness. Percent-coefficient of variation (% CV) is defined as 1 standard deviation divided by the mean, expressed as a percentage. In a %-CV plot, the lower the CV, the more ‘precise’ (statistically speaking) is the determination of the X-ray fluences of the Rh and Mo X-rays, and, consequently, the more precise the determination of the corresponding contrast values. Because the Rh and Mo X-ray spectra consist of individual X-ray photons that produce individual electrical charges in each detector element, the resulting contrast values have statistically random uncertainties associated with them, generally governed by ‘Poisson’ statistical theory. The %-CV of a CEESM contrast value is derived from the last equation shown in illustration 6. The %-CV for single-energy Mo contrast is derived by setting Φ₂ and Φ₄ to zero, while the %-CV for single-energy Rh contrast is derived by setting Φ₁ and Φ₂ to zero. Illustration 8g shows that as breast thickness increases, %-CV increases. This occurs because as the X-rays need to penetrate greater thicknesses of breast tissue, the non-characteristic higher-energy X-rays that contribute little to contrast, start to dominate the Rh and Mo spectra. Over most of the breast thickness range, the CEESM and Mo %-CV curves are essentially superimposed, with the Rh %-CV curve being consistently lower. These results suggest that the Mo X-ray exposure should be selectively increased relative to the Rh X-ray exposure. This would lower the Mo curve towards the Rh curve, which would reduce the %-CV of the CEESM contrast curve leading to a more precise determination of CEESM contrast at the lowest possible breast dose.

FIG. 8H shows %-CV vs. air KERMA. ‘Air KERMA’ (kinetic energy released in matter) may be loosely understood as the breast dose at the location of the breast, but with the breast absent, such that no scattering or absorption of the X-ray beam by the breast occurs. Air KERMA is the simplest way to incorporate breast dose as a parameter without invoking many additional variables. In illustration 8h, the CEESM and Mo %-CV curves lie virtually on top of each other with the Rh curve below them. As air KERMA increases (i.e., more X-ray photons are produced by the individual Rh and Mo X-ray exposures), the %-CV decreases, causing all three calculated contrast values to have better precision. This relationship exists with virtually every type of diagnostic imaging. In fact, in the digital imaging era, the dose determined for a specific imaging procedure is determined by balancing the risk of radiation dose to the patient vs. the diagnostic benefit incurred through improved image quality. Image quality, in this context, is determined by both contrast and by the %-CV; higher image quality corresponds to higher contrast and lower %-CV. To put illustration 8h into a more practical context, the vertical line that is shown corresponds to an air-KERMA value of 6 mGy. For a 4.2 cm breast (specified by the FDA as the ‘average compressed breast thickness’), this corresponds to an average glandular dose (AGD) of 1 mGy. The FDA, which governs radiation doses in mammography, dictates that the AGD from a single-view mammogram delivered to a standardized 4.2 cm thick breast phantom must not exceed 3 mGy. To start with, illustration 8h shows, as did illustration 8g, that either the Mo X-ray exposure should be increased or the Rh X-ray exposure decreased, to bring the Mo %-CV and Rh %-CV curves closer together, since to optimize the %-CV of the CEESM image, each individual exposure should contribute approximately equal %-CV. In addition, a specific feature of using Mo as a contrast agent, FIG. 8h also shows that a reduction in air KERMA (i.e., breast radiation dose) of 40-50% impacts the %-CVs very minimally. This means that the dose ‘penalty’ of CEESM mammography (i.e., having to deliver two X-ray exposures instead of one) could be more than offset without compromising image quality by 1) reducing the AGDs of the Mo and Rh X-ray exposures by 40-50%, and 2) raising the kVp to 45 kVp or even higher.

The mathematical task of optimizing each of the variable parameters simultaneously is a very complex one and beyond the scope of this patent application. However, because in the present parametric analysis the contrast of the CEESM technique consistently exceeds the contrast of the individual single-energy Rh and Mo X-ray techniques, one can assert with confidence that were multi-parametric optimization carried out, the contrast values for CEESM relative to single-energy Mo and Rh exposures would be at least as high than the present parametric model predicts.

### Example 3: Experimental Validation of the CEESM Technique

To determine whether the theoretical predictions of the CEESM model could be replicated in practice, a classical ‘Rose Phantom’ (12) was constructed. A Rose phantom is designed to characterize an imaging system in its ability to visually detect an object superimposed on a background using three image parameters: object diameter, object contrast, and quantum noise. As the contrast of an object to its background is progressively reduced, a threshold contrast is reached beyond which the human eye can no longer discriminate the object from its background. However, with the support of digital manipulation, it is possible to digitally amplify the object-to-background contrast, and consequently to push the threshold of detectability to still lower contrast values. But, in the presence of quantum noise (that causes statistical uncertainties in the measured signals emerging from each detector element), there is a limit to how far such digital contrast enhancement can be implemented, since at a certain point the quantum noise exceeds the average contrast between object and background, and thus further amplification of the contrast does not improve detectability of the object. As a rule-of-thumb, when the threshold contrast value approaches the standard deviation of the noise, further digital contrast amplification yields no significant improvement in object detectability since an object becomes ‘buried’ in the noise of the background. However, some further lowering of the contrast detectability threshold is still possible by increasing the size of the lesion; this permits the human eye to more easily average the quantum noise over the area of the object which allows the object to stand out from the background quantum noise.

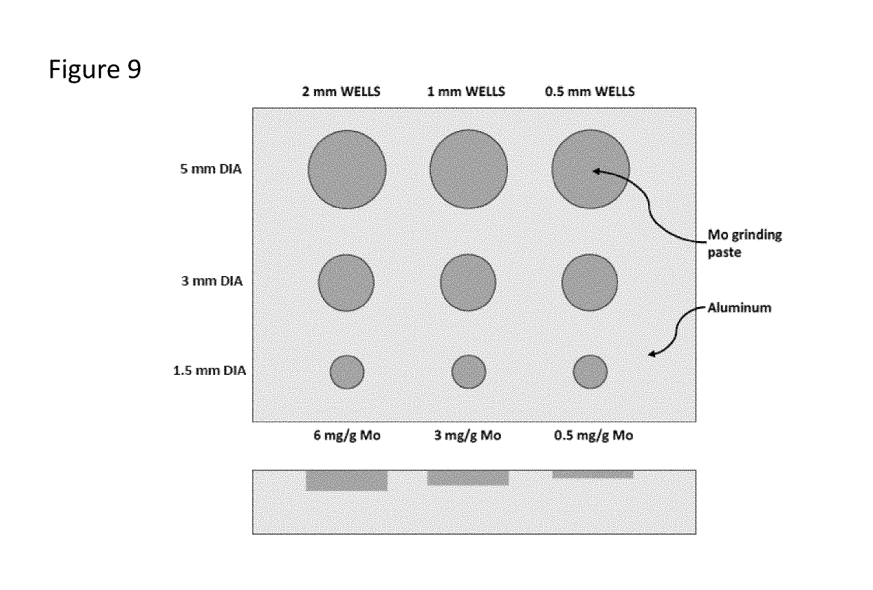

A Rose phantom was designed and constructed using a 1.27 cm (0.5 inch) thick block of aluminum, as shown in FIG. 9. The slab of aluminum contains nine milled ‘wells’ of three different diameters and three different depths (as measured along the X-ray beam axis). The three well diameters are 5 mm, 3 mm, and 1.5 mm, and the three well depths are 2 mm, 1 mm, and 0.5 mm. These parameters encompass the range of soft-tissue ‘lesion’ characteristics frequently encountered in mammography. The wells are filled with a commercial molybdenum grinding paste (Loctite 234227 L0051048 Moly Paste), which serves to emulate the range of concentrations of Mo modeled in the breast tumor. The three wells of varying depth provide Mo concentrations of 6, 3, and 1 mg/g. The three different well diameters may be considered as representing three different sized breast tumors.

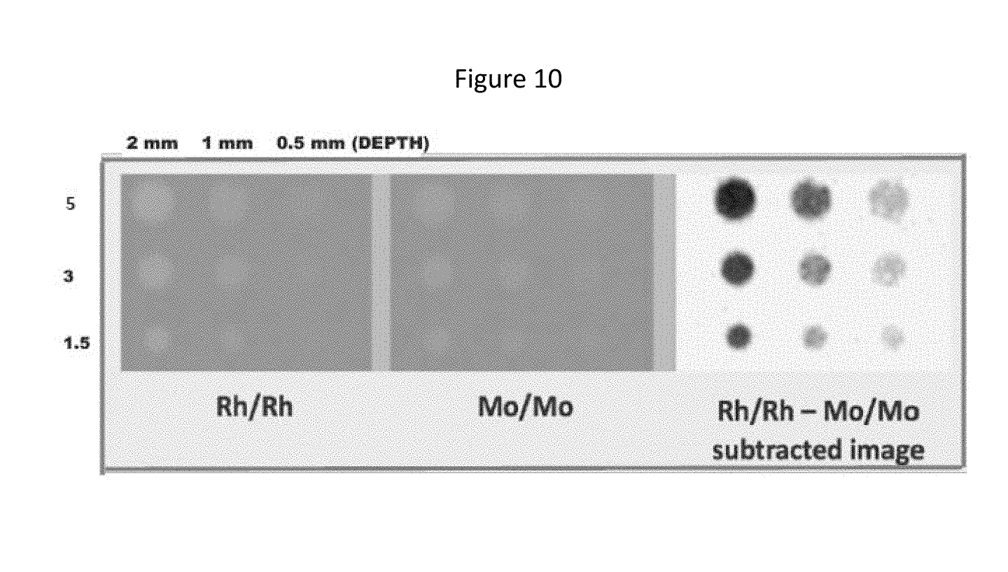

FIG. 10 shows radiographs of the Rose phantom obtained using a full-field digital General Electric mammography machine having the capability of generating both Mo and Rh X-rays. The exposure factors used were 30 kVp and 100 mAs with the ‘large’ (0.3 mm) focal spot. The Mo and Rh images were digitally subtracted using Adobe Photoshop CS3. The left-most panel of FIG. 10 is a radiograph of the Rose phantom using the Rh X-ray spectrum filtered by 0.03 mm of Rh; the middle panel is a radiograph of the Rose phantom using a Mo X-ray spectrum filtered by 0.03 mm of Mo; and the right-most panel shows the CEESM subtraction image. The improved contrast and detectability of all nine Mo-filled wells in the CEESM image is clearly evident. Also of note is that Mo ‘tumor’ concentrations as low as ˜1 mg/g are clearly detectable in the CEESM image.

These images represent the first ever experimental demonstration of the CEESM principle.

### Example 4: Evaluating the CEESM Technique Clinically

In the process of obtaining dual-energy CEESM mammographic images, it should be emphasized that single-energy Mo and Rh images would still be available for independent evaluation. Therefore, no information that is produced by current conventional mammography would be lost when implementing the CEESM technique. This fact is crucially important when a new imaging technique is introduced so that correlations can be established between the ‘old’ and the ‘new’ imaging techniques with negligible increases in radiation dose.

In the case of a preliminary clinical CEESM study, Rh and Mo X-ray mammograms (i.e., ‘conventional’ mammograms) and CEESM mammograms would be simultaneously available for direct display and comparison, and if the X-ray exposures for the Mo and Rh images were halved, no additional dose to the breast would be incurred and all segments of the screening population would, therefore, be admissible to such investigatory studies. A number of maneuvers can be effected to halve the AGD with little or no adverse impact on image quality. For example, higher kVp and/or lower air KERMA can be implemented (see discussion of FIGS. 8H and 8C).

### Example 5: Theoretical Calculation of the Relative Contrast Values for Iodine an Molybdenum

In Lewin (10), a dual-energy mammographic machine manufactured by Lorad, Inc. was modified to produce Mo and Rh X-ray exposures in rapid succession to enable contrast-enhanced dual-energy imaging to be done as an experimental study. The contrast agent was conventional iodine in the form of Iohexole. The conclusions of this study were that contrast-enhanced dual-energy imaging with Iohexole was a useful development in mammography. Since the technical characteristics of the mammography equipment used were documented in this study, it provides the opportunity of a useful comparison with the CEESM technique. The high-energy exposure used a Rh X-ray spectrum filtered with a 0.025 mm Rh filter and an 8 mm aluminum filter. The low energy exposure used a Mo X-ray spectrum filtered with a 0.03 mm Mo filter. The iodine-based contrast agent used was Iohexole.

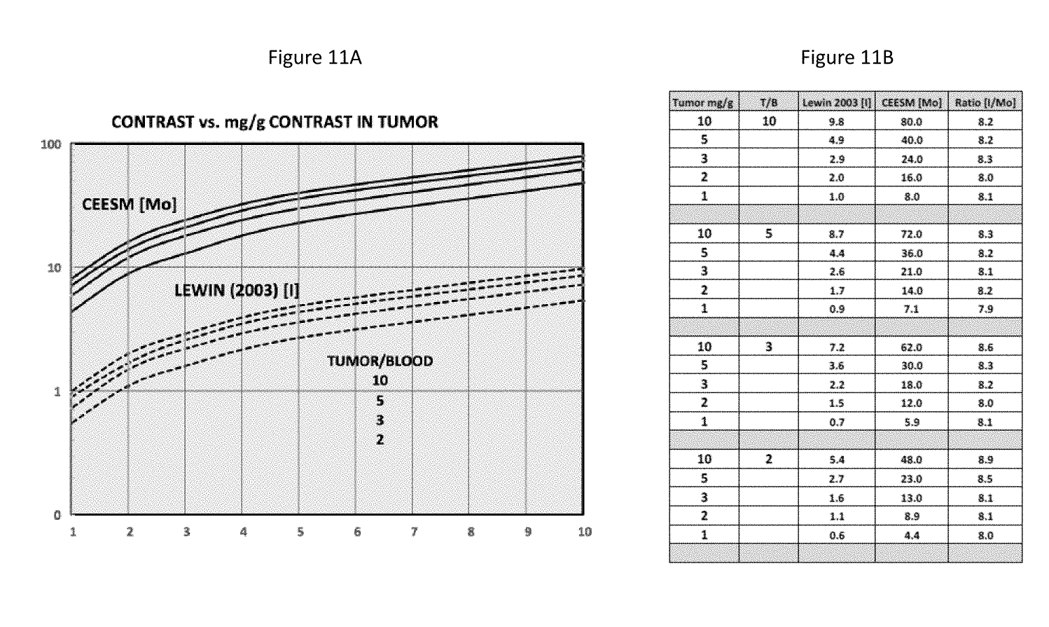

FIG. 11 shows the results of the comparison between the CEESM technique (using a Mo-based contrast agent) and the technique reported in Lewin using an iodine-based contrast agent. The concentration of the two contrast agents was normalized on the basis of equal molecular masses of Mo and iodine. FIG. 11 clearly shows that, regardless of tumor/blood ratio or tumor contrast concentration assumed, the CEESM technique produces contrast values that are about 7-8 times higher than the technique used in Lewin.

### CITED REFERENCES

1. 1999-2014 CDC Incidence and Mortality Web-based Report
2. World J Clin Oncol 2014; 5(2): 61-70
3. Breast Care Basel 2010; 5(2):109-114
4. AJR 2014; 202:299-308
5. Asian J of Pharma Sci 2017; 12:235-249
6. J of Inorg Biochem 2015; 144:13-17
7. Chem Rev 1999; 99:2353-2377
8. PLoS ONE 2016; 11(9):e0162740
9. European Radiology 2011; 21(3):565-574
10. Radiology 2003; 229(1):261-268
11. Med Phys 2017; 44(6):2148-2160
12. J Opt Soc Am 1946; 38:196-208
13. Med Phys 1981; 8:629-639
14. Phys Med Biol 1991; 36:1165-1182
15. AJR 1994; 162:1313-1317
16. Circulation 2007; 115:1211-1217
17. United States Public Health Service, Public Health Bulletin; 1945:293

### INCORPORATION BY REFERENCE

All U.S. patents and U.S. and PCT published patent applications mentioned in the description above are incorporated by reference herein in their entirety.

### EQUIVALENTS

Having now fully described the present invention in some detail by way of illustration and examples for the purposes of clarity of understanding, it will be obvious to one of ordinary skill in the art that the same can be performed by modifying or changing the invention within a range of conditions, formulations and other parameters without affecting the scope of the invention or any specific embodiment thereof, and that such modifications or changes are intended to be encompassed within the scope of the appended claims.

We claim:

1. A method of dual energy X-ray imaging, comprising the steps of:
administering to a subject an effective amount of a molybdenum-based contrast agent; wherein said administration is intravenous;
acquiring a first image of a tissue of the subject with a low energy spectrum; and
acquiring a second image of the tissue of the subject with a high energy spectrum.

2. The method of claim 1, wherein the molybdenum-based contrast agent is molybdenum disulfide.

3. The method of claim 1, wherein the contrast agent is administered to the subject in an amount sufficient to produce a concentration of about 0.3-3 mg of molybdenum/g.

4. The method of claim 1, wherein the low energy spectrum comprises a low energy spectrum filtered with a molybdenum filter.

5. The method of claim 1, wherein the high energy spectrum comprises a high energy spectrum filtered with a rhodium filter.

6. The method of claim 1, wherein the low energy spectrum and high energy spectrum are both acquired within less than 100 milliseconds elapsed time.

7. The method of any one of claim 1, wherein the low energy spectrum and high energy spectrum are both acquired within less than 10 milliseconds elapsed time.

8. A method of multi-mode X-ray examination of a contrast-enhanced patient's breast, comprising the steps of:
introducing intravenously a vascular contrast agent into a patient's breast; wherein the contrast agent is a molybdenum-based contrast agent;
obtaining (i) a low-energy X-ray 2D mammogram of the breast, and (ii) a high-energy X-ray 2D mammogram of the breast;
wherein said low-energy and high-energy 2D mammograms are taken in a single breast compression;
computer-processing the 2D low-energy mammogram and the 2D high-energy mammogram to form a weighted combination 2D image highlighting vascularity in the breast; and
displaying the weighted combination 2D image;
wherein the displayed weighted combination 2D image facilitates identification of a position of a possible abnormality in two dimensions.

9. The method of claim 8, further comprising obtaining a multiplicity of single-energy X-ray 2D tomosynthesis projection images of the patient's breast; computer-processing the 2D tomosynthesis projection images into 3D slice images that represent respective slices of the breast; and displaying the weighted combination 2D image and one or more of the 3D slice images; wherein the one or more 3D slice images facilitate identification of a position of the abnormality in three dimensions and visualization of the appearance of the abnormality in respective slice images.

10. The method of claim 9, wherein the multiplicity of single-energy X-ray 2D tomosynthesis projection images are acquired prior to acquiring the low-energy and high-energy X-ray 2D mammograms.

11. The method of claim 8, wherein said displaying comprises concurrently displaying the combination 2D image and at least one of the 3D slice images.

12. The method of claim 8, wherein said displaying comprises displaying the combination 2D image and at least one of the 3D slice images in mutual registration.

13. The method of claim 8, wherein said displaying comprises concurrently displaying the combination 2D image and a plurality of the 3D slice images.

14. The method of claim 8, wherein said displaying comprises concurrently displaying the combination 2D image and a subset of the 3D slice images, wherein the subset comprises 3D slice images in which the abnormalities appear and the subset is computationally selected in response to an identification of the abnormality in the combination 2D image.

15. A method of multi-mode X-ray examination of a contrast-enhanced patient's breast, comprising the steps of:
introducing intravenously a vascular contrast agent into a patient's breast; wherein the contrast agent is a molybdenum-based contrast agent;
obtaining (i) a multiplicity of single-energy X-ray 2D tomosynthesis projection images of the breast, (ii) a low-energy X-ray 2D mammogram of the breast, and (iii) a high-energy X-ray 2D mammogram of the breast; wherein said 2D tomosynthesis projection images are obtained before said 2D mammograms, and the 2D tomosynthesis projection images and the mammograms are taken in a single breast compression;
computer-processing the 2D low-energy mammogram and the 2D high-energy mammogram to form a weighted combination 2D image highlighting vascularity in the breast; and
displaying the weighted combination 2D image and one or more of the 3D slice images; wherein the displayed weighted combination 2D image facilitates identification of a position of a possible abnormality in two dimensions, and the displayed one or more 3D slice images facilitate identification of the position of the abnormality in three dimensions and visualization of appearance of the abnormalities in respective slice images.

16. The method of claim 15, further comprising obtaining 3D slice images of the patient's breast that represent respective slices of the breast and are reconstructed through computer-processing a multiplicity of single-energy X-ray 2D tomosynthesis projection images.

17. The method of claim 15, wherein the 3D slice images of the patient's breast are obtained prior to acquiring the low-energy and high-energy X-ray 2D mammograms.

18. The method claim 15, wherein said displaying comprises concurrently displaying the weighted combination 2D image and at least one of the 3D slice images.

19. The method of method claim 15, wherein said displaying comprises displaying the weighted combination 2D image and at least one of the 3D slice images in mutual registration.

20. The method of method claim 15, wherein said displaying comprises concurrently displaying the weighted combination 2D image and a plurality of the 3D slice images.

21-32. (canceled)
